(19) United States
(12) Patent Application Publication
(10) Pub. No.: US 2025/0319322 A1
(43) Pub. Date: Oct. 16, 2025

(54) Body Cavity Light Based Treatment Devices

(71) Applicant: Designs for Vision, Inc., Bohemia, NY (US)

(72) Inventor: Richard E. Feinbloom, New York, NY (US)

(21) Appl. No.: 18/697,462

(22) PCT Filed: Apr. 7, 2023
(86) PCT No.: PCT/US2023/017924

§ 371 Date: Apr. 4, 2024

Related U.S. Application Data
(63) Continuation-in-part of application No. PCT/US2022/045643, filed on Oct. 4, 2022.
(60) Provisional application No. 63/252,724, filed on Oct. 6, 2021.

(51) Int. Cl. A61N 5/06

(52) U.S. Cl. CPC A61N 5/0603; A61N 5/0625; A61N 2005/0643; A61N 2005/0667

(57) ABSTRACT

A body cavity treatment device including a plurality of lighting sources that are configured to provide for the detection of infection and the treatment of the detected infection, wherein the lighting sources are positioned substantially adjacent to the detected infection when the treatment device is inserted into the body cavity. In one aspect, an active cooling device is incorporated to maintain consistent temperature of the device within the body cavity.

### CLAIM OF PRIORITY

This application claims, as a Continuation-in-part application, pursuant to 35 USC 120, priority to, and the benefit of the earlier filing date of that patent applications, filed in the US Receiving Office on Oct. 4, 2022 and afforded serial number PCT/US2022/045643, which claimed, pursuant to 35 USC 119, priority to, and the benefit of the earlier filing date of that provisional patent application filed in the US Patent and Trademark Office on Oct. 6, 2021 and afforded Ser. No. 63/252,724, the contents of which are incorporated by reference, herein.

### BACKGROUND OF THE INVENTION

### Field of the Invention

This invention relates to the field of medical examination/diagnosis and treatment and more particularly to a devices utilizing Fluorescent Enhancement Theragnosis technology in the identification and treatment of medical conditions related to a body cavity, e.g., an oral cavity, anus and vagina.

### Background Information

Light based therapy has emerged as a useful tool in locating, identifying and treating infection or bacterial load within bodily tissues to reduce or eliminate the bacterial load within the tissues. Light based therapy is intended to provide for photoactivated disinfection, photodynamic therapy and photobiomodulation.

Photoactivated therapy is based on the use of inactive prodrugs whose biological activity is significantly increased upon exposure to light. See, for example, F. Reeßing, W. Szymański: Beyond Photodynamic Therapy: Photoactivated Cancer Chemotherapy, Curr. Med. Chem., 2018, 24, 4905-4950.

Photoactivated disinfection (PAD) is based on the concept of photodynamic therapy (PDT). PDT is a treatment method that is associated with the use of a light source and a photosensitizer (PS) that binds to cells and after absorption of light induces a chemical reaction that results in the release of radicals and singlet oxygen. See, for example, Husejnagic S, Lettner S, Laky M, Georgopoulos A, Moritz A, Rausch-Fan X. Photoactivated disinfection in periodontal treatment: A randomized controlled clinical split-mouth trial. J Periodontol. 2019; 90:1260-1269.|

Photobiomodulation (PBM) is a non-invasive method that contributes to pain relief and reduces inflammation, parallel to the enhanced healing and tissue repair processes. See, for example, Dompe, C.; Moncrieff, L.; Matys, J.; Grzech-Leśniak, K.; Kocherova, I.; Bryja, A.; Bruska, M.; Dominiak, M.; Mozdziak, P.; Skiba, T. H. I.; Shibli, J. A.; Angelova Volponi, A.; Kempisty, B.; Dyszkiewicz-Konwińska, M. Photobiomodulation-Underlying Mechanism and Clinical Applications. J. Clin. Med. 2020, 9, 1724.

The referred to research has found that the application of light within known wavelength ranges when applied to a surface is an effective means for reducing the activity of virus and bacteria. Such research, referred to, herein, as Fluorescent Enhancement Theragnosis (FET) technology, has proved itself to provide benefit to practitioners, such as dentist, doctors and surgeons, etc., in identifying bacteria that would not normally be viewable by the practitioner.

For example, FET has been found use in dental practice to apply light to a patient's mouth to identify virus and/or bacteria and reduce the activity of the identified virus/bacteria and, hence, reduce the likelihood of the patient contracting an infection from the virus or bacteria.

Similarly, FET technology provides a doctor or surgeon the ability to view virus and/or bacteria on or within a patient and initiate appropriate remedial actions when necessary.

However, the light utilized in FET technology may be harmful to the human eye and, thus, eye protection is required for both the patient and the practitioner to avoid either party from inadvertently viewing the harmful light. In addition, the lighting sources used further generate significant heat, which prevents the devices from being retained within the body cavity for extended periods of time.

Hence, there is a need in the industry for applying a light therapy to a patient while preventing both the burning of the patient and the inadvertent viewing of the applied light.

### SUMMARY OF THE INVENTION

Disclosed are devices to apply light therapy to infection such as bacteria and viruses within a body cavity of a patient.

In one aspect of the invention, a light therapy device comprising a plurality of lighting sources provides for the distribution of light into a body cavity, such as the mouth, the anus, or the vagina, wherein the lighting elements provide for the detection of infection within the body cavity, for example, and for the application of light thereto.

In one aspect of the invention, a light therapy device comprising a plurality of lighting sources positioned on a light bearing device attached to a probe element provides for the distribution of light that may be used for the identification and the treatment of infection within the body cavity.

In one aspect of the invention, a light therapy device comprising a plurality of lighting sources on a light bearing device attached to a probe element provides for the distribution of a light treatment substantially adjacent an area within the body cavity identified as having bacterial or viral infection while allowing for the prolonged placement of the device in the body cavity by providing a cooling mechanism to draw heat from the body cavity.

For a better understanding of exemplary embodiments and to show how the same may be carried into effect, reference is made to the accompanying drawings. It is stressed that the particulars shown are by way of example only and for purposes of illustrative discussion of the preferred embodiments of the present disclosure and are presented to clarify the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings:

FIG. 1A illustrates an exploded prospective view of a first exemplary embodiment of a light therapy device for applying light therapy to a body cavity in accordance with the principles of the invention.

FIG. 1B illustrates an exemplary configuration of the light arrays providing light therapy in accordance with the principles of the invention.

FIG. 2 illustrates a front view of a first aspect of the light therapy device shown in FIG. 1A.

FIG. 3 illustrates a side view, through section B-B, of the light therapy device shown in FIG. 2.

FIG. 4 illustrates an expanded view of the area designated as FIG. 4 shown in FIG. 3.

FIG. 5 illustrates a front view of the light treatment device shown in FIG. 1A.

FIG. 6 illustrates a side view, through section C-C, of the light therapy device shown in FIG. 5.

FIG. 7 illustrates an expanded view of the area designated as FIG. 7 shown in FIG. 6.

FIG. 8 illustrates an exploded prospective view of a second exemplary embodiment of a light therapy device in accordance with the principles of the invention.

FIG. 9 illustrates an expanded prospective view of the area designated as FIG. 9 in FIG. 8.

FIG. 10 illustrates a prospective view of the assembled second exemplary embodiment shown in FIG. 8.

FIG. 11 illustrates a prospective view of another aspect of the second exemplary embodiment shown in FIG. 8.

FIG. 12 illustrates a side view of the second exemplary embodiment of a light therapy device shown in FIG. 8.

FIG. 13 illustrates a side view of another aspect of the exemplary embodiment of the light therapy device shown, herein.

FIG. 14 illustrates a front view exemplary embodiment of the light therapy device shown in FIG. 13.

FIG. 15 illustrates an exploded prospective view of a second aspect of the first exemplary embodiment of the light therapy device shown in FIG. 1A.

FIG. 16 illustrates a side view of the light therapy device shown in FIG. 15.

FIG. 17 illustrates an exploded prospective view of a second aspect of the second exemplary embodiment of the light therapy device shown in FIG. 8.

FIG. 18 illustrates a side view of the light therapy device shown in FIG. 17.

FIG. 19 illustrates a prospective view of the light therapy device shown in FIG. 19.

FIG. 20 illustrates a prospective view of an exemplary system for operating the light therapy device shown, herein.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION OF THE INVENTION

As used herein, the terms “comprises”, “comprising”, “includes”, “including”, “has”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “of” refers to an inclusive “or” and not to an exclusive “or”. For example, a condition A or B is satisfied by any one of the following: A is true (or present) and B is false (or not present); A is false (or not present) and B is true (or present); and both A and B are true (or present).

The terms “a” or “an” as used herein are to describe elements and components of the invention. This is done for convenience to the reader and to provide a general sense of the invention. The use of these terms in the description, herein, should be read and understood to include one or at least one. In addition, the singular also includes the plural unless indicated to the contrary. For example, reference to a composition containing “a compound” includes one or more compounds. As used in this specification and the appended claims, the term “or” is generally employed in its sense including “and/or” unless the content clearly dictates otherwise.

All numeric values are herein assumed to be modified by the term “about,” whether or not explicitly indicated. The term “about” generally refers to a range of numbers that one of skill in the art would consider equivalent to the recited value (i.e., having the same function or result). In any instances, the terms “about” may include numbers that are rounded (or lowered) to the nearest significant figure.

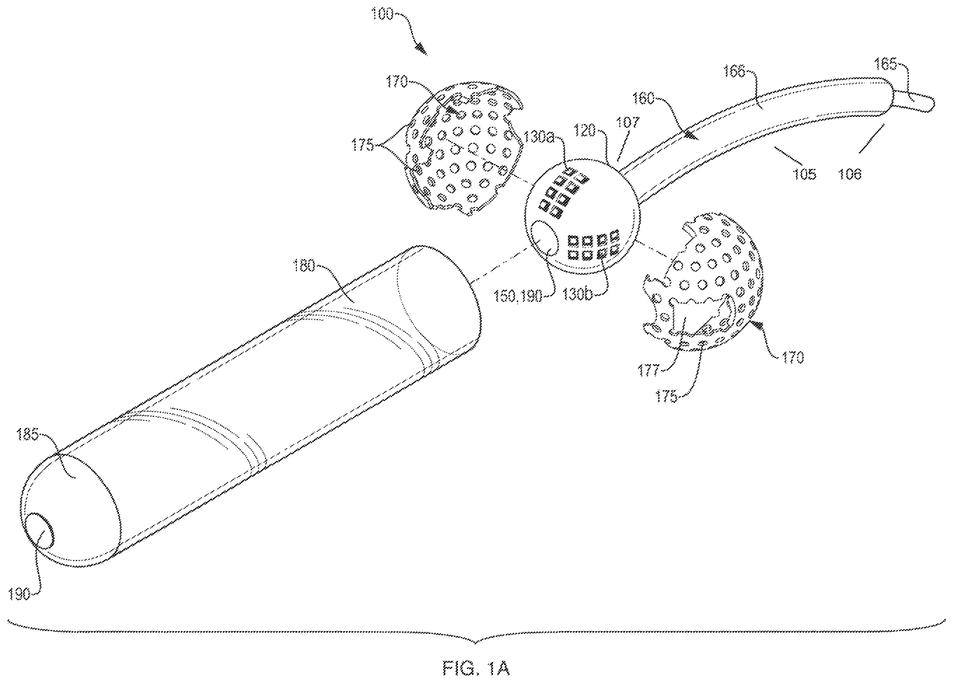

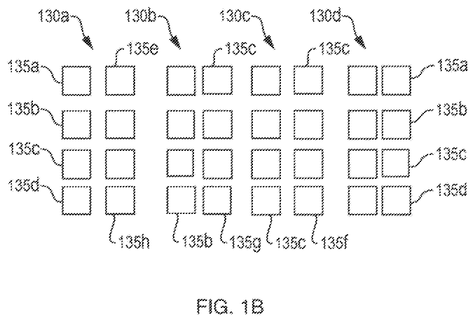

FIG. 1A illustrates an exploded prospective view of a first exemplary embodiment of a light therapy device in accordance with the principles of the invention.

Light therapy device 100 comprises an elongated probe element 105 extending from a proximal end 106 to a distal end 107, wherein a light bearing element 120 is positioned at distal end 107 of probe element 105.

In one aspect of the invention, light bearing element 120 may be integrated onto the distal end 107 of probe element 105. Alternatively, light bearing element 120 may be removably attached to distal end 107 of probe element 105.

Further illustrated is sheathing 160 extending from proximal end 106 to distal end 107. Sheathing 160 incorporates an electrical connector element 165, therein. Connector element 165 provides a means for transferring control and power signals from a power source (not shown) to an electrical or electronic circuit (not shown) positioned within the light bearing element 120. Although not shown, it would be recognized that the not shown electrical circuitry may include electrical elements such as resistors, capacitors, transistors, integrated circuits, that are well-known in the art and thus, a detailed discussion of these components is believed not necessary for those skilled in the art to practice the invention claimed.

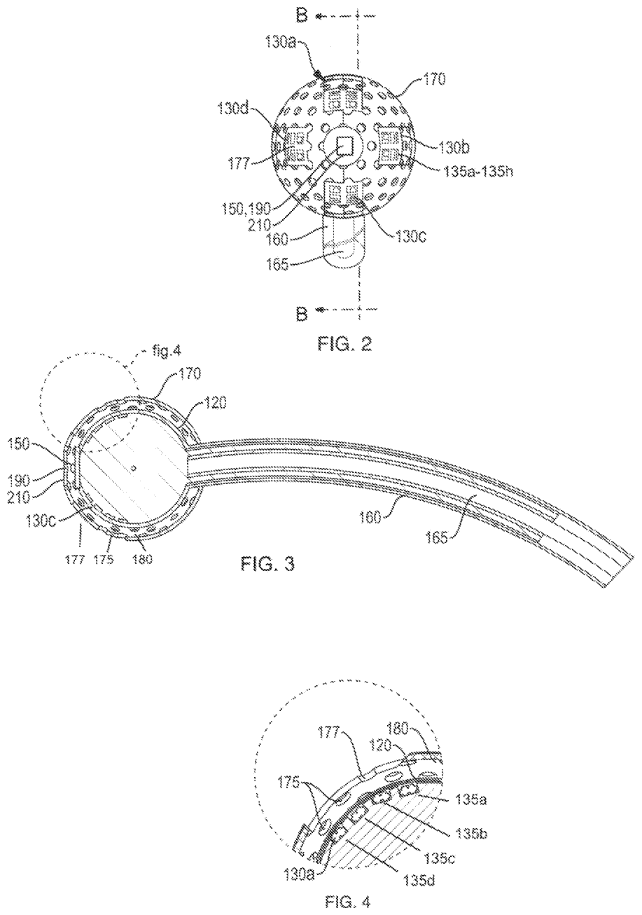

Lighting arrays 130 (of which two are shown and referred to as 130a, 130b) positioned in an orthogonal relationship on an outer surface of light bearing element 120 comprises a plurality of lighting sources (see FIG. 2) that generate light in one or more wavelength ranges. For example, the lighting sources may generate or emit light in one or more of an ultra-violet wavelength range, a visible wavelength range, which may be a white (i.e., 380-750 nm) or one or more of a color light (e.g., violet—380-435 nm; blue—435-495 nm; cyan—495-520; green—420-570 nm; yellow—570-590 nm; orange—590-620 nm and red—620-750 nm) and an infra-red wavelength range. The light emitted by the illustrated lighting arrays 130a, 103b may be emitted individually or in combination.

Although two lighting arrays (130a, 130b) are shown, it would be recognized that with the illustrated orthogonal positioning of light arrays 130a, 130b, at least two additional light array 130a-130d (not shown) may be positioned along a surface of light bearing element 120, such that a total of four (4) light arrays may be positioned on light bearing element 120. In addition, it would be understood that the number of light arrays 130 may be further increased based on the selection of a different positional relationship of the illustrated light arrays 130.

In accordance with one aspect of the invention, sheathing 160 may further comprise a flexible, pliable or a semi-rigid material that provides protection from the electrical energy provided through electrical connector 165. In addition, sheathing 160 may contain therein, a heat conductive path 166 (i.e., embedded heat conductive elements, e.g., aluminum, cooper) through which heat generated by the operation of lighting arrays 130 on ball element 120 may be drawn from distal end 107 to proximal end 106. In one aspect of the invention, the heat conductive path may be represented as electrical connections 165. Alternatively, heat conductive path 166 may be represented as a separate entity incorporated into sheathing 160.

In accordance with the principles of the invention, each of light arrays 130 (130a, 130b, etc.) may comprise a plurality of lighting sources 135a, 135b, etc., (see FIG. 1B) which, in this exemplary illustration, are shown as eight (8) lighting sources. The illustrate lighting sources may comprise one or more light emitting diode (LEDs) that emit light in one or more known wavelength range. For example, the illustrated lighting sources may emit light in a same or different wavelength range, where the number of lighting sources emitting light at a substantially same wavelength increases the intensity of the light emitted.

FIG. 1B illustrates an exemplary configuration of the lighting sources 135a-135h in each of the illustrated lighting arrays 130a, 130b, shown in FIG. 1A, and in the not shown lighting arrays 130c, 130d. In these illustrated exemplary configurations, a same lighting source 135a-135h is shown in different positions within each of lighting arrays 130a-130d, wherein a different positioning of the lighting sources provides for a wider distribution of light emitted by a same lighting source 130a-130h.

Although FIG. 1B illustrates different light sources 135a-135h at different positions within different light arrays 130a, 130b, etc., it would be obvious to those skilled in the art that the position of light sources 135a-135d within each of light arrays 130a-130d may be the same or altered from the positions shown in FIG. 1B, without altering the scope of the invention claimed. In addition, although different light sources 135a-135h are illustrated, it would be obvious to those skilled in the art, that each of light sources 135a-135h in each of light arrays 130a-130d may emit a substantially same light wavelength.

Returning to FIG. 1A, further illustrated is an image capture element (e.g., camera, charged coupled device sensor, TV camera, CMOS image sensor, etc.) 150 positioned on a distal end of light bearing element 120. Image capture element 150 provides for the collection of light emitted by light sources 135a-135h that may be reflected by an object (e.g., tissue) within a body cavity into which device 100 is inserted, illuminated by the emitted light. In one aspect of the invention, image capture element 150 provides for the collection of light generated (i.e., a fluorescent light) by the interaction of the light emitted by light sources 135a-135h with body cavity tissue in response to being illuminated by the light emitted by light sources 135a-135h.

As is known in the art, bacterial and viral infection may self-generate a light (i.e., fluorescent light) when illuminated by known wavelengths, wherein the self-generated light provides a practitioner the ability to distinguish healthy tissue from diseased (or bacteria laden) tissue.

Electrical representation (i.e., pixels) associated with the images captured by image capture device 150 may be transmitted through cable 165 to an electronic circuitry (not shown). The not shown electronic circuitry, which may be composed of well-known electrical components (e.g., ASIC), may further transmit the receive pixels to a storage device (not shown) or a viewing screen (not shown), such as a TV monitor or computer monitor.

In this exemplary embodiment, light therapy device 100 may further include a filter (hereinafter referred to as emission filter) 190 positioned adjacent to, and in front of, image capture device 150. Emission filter 190 is configured to limit the wavelength range viewed or captured by image capture device 150. For example, emission filter 190 may comprise optical material that limits the range of wavelengths that are viewable by image capture device 150 to be within wavelength ranges comparable to a fluorescent light self-generated by a bacteria and/or viral infection when illuminated by light emitted by one or more of lighting sources 135a-135h.

In one aspect of the invention, the optical material characteristics, which may be of an absorptive nature or a reflective nature, of emission filter 190 may remove or significantly attenuate the light emitted by lighting sources 135a-135h, such that light emitted by lighting sources 135a-135h is not captured by image capture device 150. For example, light emitted in at a wavelength emitted by one or more of lighting sources 135a-135h, may be significantly reduced in amplitude or intensity when viewed through filter 190. But other light, when viewed through filter 190 may be viewed without any attenuation.

Further illustrated is probe (or device) sleeve 180 into which light bearing element 120 may be contained. Probe sleeve 180, which may be composed of a flexible material, provides for the smooth and non-abrasive insertion of light therapy device 100 into a body cavity of a patient in a safe and sanitary manner.

Probe Sleeve 180 may be removable attachable to probe 105 to provide for the proper cleaning of sleeve element 180 or to be disposable. In addition, sleeve element 180 may be a clear (transparent) material, a translucent material, or a frosted material, wherein light passing through a translucent or frosted sleeve 180 is diffused.

In one aspect of the invention, emission filter 190 may be positioned on distal end 185 of probe sleeve 180 rather than adjacent to image capture device 150, as shown. In this aspect of the invention, distal end 185 may comprise optical material, similar to that discussed above, that limits the wavelength range viewed by image capture device 150, as previously discussed.

In accordance with another aspect of the invention, a light distributor 170 may be incorporated into light therapy device 100, into which light bearing element 120 may be positioned.

In one aspect of the invention, light distributor 170, as shown, may comprise a plurality of perforations (or holes) 175 that allow light emitted by one or more of lighting sources 135a-135h to pass through to illuminate an area (e.g., body tissue) substantially adjacent light distributor 170. Similar to sleeve element 180, sphere 170 may be removably attached to light bearing element 120 to provide for the proper cleaning of light distributor 170 or to be disposable.

In another aspect of the invention, light distributor 170 may be composed of an optically clear, optically clear in designated area, translucent and/or frosted material that enables light to pass through to body tissue substantially adjacent light bearing element 120. In still another aspect of the invention, light distributor 170 may be composed of an optically opaque material into which perforations 175 reside. Light emitted by lighting sources 135a-135h may exit light distributor 170 through perforations 175.

Although light distributor 170 is shown perforated with a plurality of holes or circular openings 175, it would be recognized by those skilled in the art that the perforations within light distributor 170 may comprise slots, which may be vertically or horizontally, with respect to a horizontal axis of light distributor 170, without altering the scope of the invention claimed.

In one aspect of the invention, light distributor 170 may include a plurality of openings 177, rather than, or in addition to, perforations 175, sized to accommodate the position of light arrays 130 on light bearing element 120. In this illustrated embodiment, openings 177 (of which only one is shown) is positioned to accommodate the lighting sources shown as light array 130b. However, it would be understood that light distributor 170 may include opening 177 for each of light arrays 130 positioned on light ball 120.

In accordance with the principles of the invention, sleeve element 180 and light distributor 170 may be sized such that light distributor 170 fits over sleeve element 180 or sleeve element 180 may be sized to fit over light distributor 170.

Although light bearing element 120 is shown as substantially spherical, it would be recognized that light bearing element 120 may an elongated, elliptical or tubal shape, with respect to a longitudinal axis of the probe element 105, wherein the shaped of light bearing element 120 allows for the smooth and non-abrasive entry of light therapy device 100 into the body cavity of a person to whom light therapy is to be applied.

For example, the characteristics of light bearing element 120 may be formed similar to the tubal shape shown with regard to sheathing 180, wherein the lighting sources are incorporated on to a distal end of the tubal shape which is similar to distal end 185. In this aspect of the invention sheathing 180 may not be needed, as the tubal shape of light bearing element 120 provides for the smooth and non-abrasive entry of probe 105 into a body cavity.

FIG. 2 illustrates a front view of a first aspect of the light therapy device 100 shown in FIG. 1A in accordance with principles of the invention.

In this illustrated view light bearing element (e.g., a sphere) 120 is enclosed within light distributor 170, which includes both perforations 175 and opening 177, wherein four openings 177 are shown to accommodate the illustrated four light arrays 130a-130d. In this illustrated aspect, light array 130b is shown comprising lighting sources 135a-135h (which is similar for light arrays 130a, 130c and 130d). Further illustrated is forward opening, or optically clear area 210, positioned on a distal end of sphere 120. Opening or optical clear area 210, which aligns with image capture device 150 and emission filter 190, provides an optical clear path for the capturing of images by image capture device 150. Alternatively, area 210 may include material that causes area 210 to operate as emission filter 190, wherein selected wavelengths of light are provided to image capture device 150. Alternatively, protective sleeve 180 (not shown) may be further incorporated into the illustrated lighting device 100, wherein area 210 aligns with distal end 185 of protective sleeve 180.

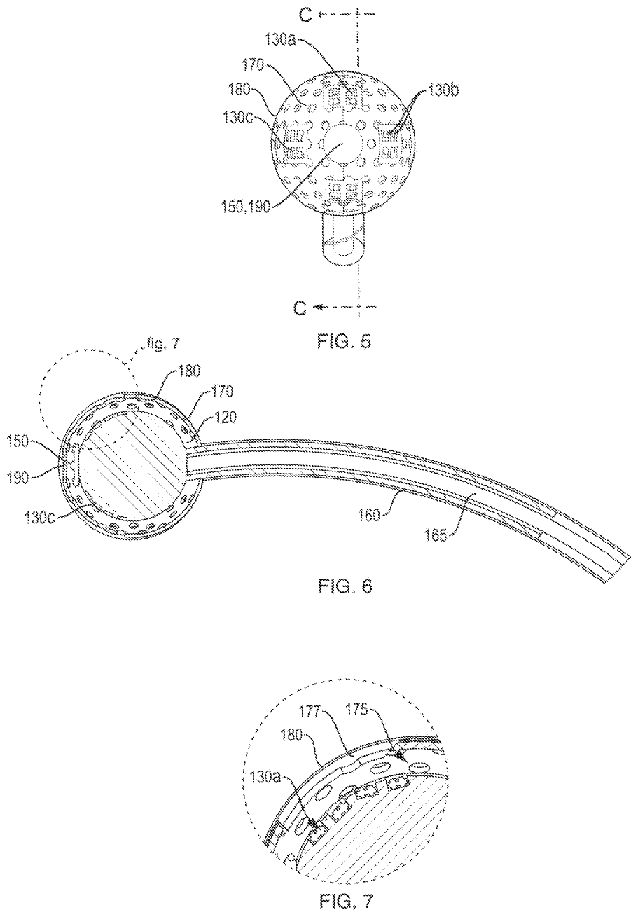

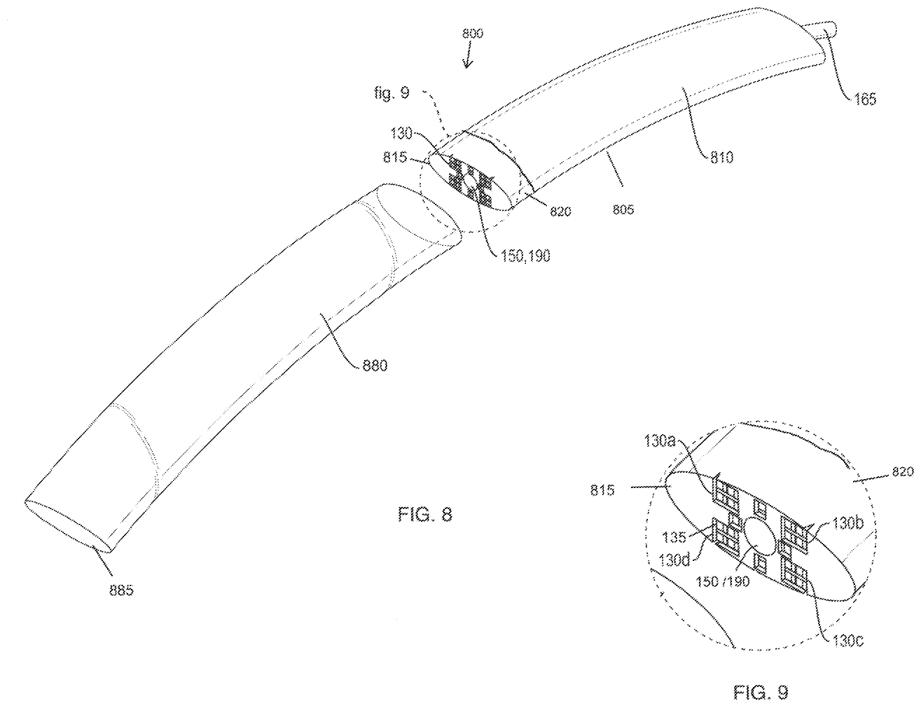

FIG. 3 illustrates a side view, through section B-B of the first aspect of light therapy device 100 shown in FIG. 2.

In this illustrated side view, light bearing element 120 is shown enclosed within sleeve element and light distributor 170 wherein both light bearing element 120 and protective sleeve 180 are shown as spherical elements. In this illustrate aspect of the invention, light distributor 170 is the outermost layer of light therapy device 100. Further illustrated is sheathing 160 through which electrical connection 165 provides power and control signals to a circuit board (not shown) that controls the application of power (electrical energy) to one or more of the lighting sources 135a-135h within respective ones of lighting arrays 130a-130d. Electrical connection 165 further provides a path for the transmission of the pixels associated with images captured by image capture device to be stored or displayed as previously discussed.

FIG. 4 illustrates an expanded view of the area identified as FIG. 4 in FIG. 3.

In this illustrated expanded view, light bearing device 120 including light array 130a comprising a plurality of lighting sources 135a-135h is configured to project light through sleeve element 180. Further illustrated is light distributor 170 positioned around the combination of light ball 120 and sleeve 180, wherein lighting sources 135a-135h are arranged to project light through sleeve 180 and/or opening 177 (and/or perforations 175).

FIG. 5 illustrates a front view of another aspect of light therapy device 100 shown in FIG. 1A.

In this illustrated second aspect, light therapy device 100 comprises light bearing element 120 encapsulated by light distributor 170, which is encapsulated within sleeve element 180, wherein light emitted by lighting sources 135a-135h, is emitted adjacent to tissue within the body cavity.

FIG. 6 illustrates a side view, through section C-C shown in FIG. 5, of the exemplary aspect of light therapy device 100.

In this illustrated side view, light bearing element 120 is shown enclosed within light distributor 170 and sleeve 180, wherein sleeve 180 is the outermost layer of light therapy device 100. Further illustrated is sheathing 160 through which an electrical connection 165 provides power and control signals to electrical circuitry (not shown) that applies power to one of more of e lighting sources 135a-135h of respective ones of lighting arrays 130a-130d.

FIG. 7 illustrates an expanded view of the area identified as FIG. 7 in FIG. 6,

In this illustrated expanded view, light bearing element 120, including light array 130a is configured to project light through opening 177 of light distributor 170 such that emitted light passes through light distributor 170/perforations 175. Further illustrated is protective sleeve 180, which is spherically shaped, positioned about the combination of light bearing element 120 and light distributer 170.

FIG. 8 illustrates a second exemplary embodiment of a light therapy device in accordance with the principles of the invention.

In this illustrated second exemplary embodiment, light therapy device 800 comprises a probe element 805, in the form of a housing 810, which is shown as possessing an elongated cross-sectional shape, and a light bearing element 820 attached to a distal end of housing 810.

In accordance with the principles of the invention, housing 810 may be formed composed of a rigid, semi-rigid, pliable or flexible material formed in a straight or curved form. For example, housing 810 may be formed utilizing a rigid material with a known curved shape. Alternatively, light therapy device 800 (i.e., housing 810) may be formed with a straight shape, wherein housing 810 includes a pliable material that allows for the alteration of the shape of light therapy device 800 into a desired curved shape.

Further illustrated is light bearing element 820 formed in a shape that is conformal to the transverse elongated cross-sectional shape of housing 810, wherein light bearing element 820 comprises a plurality of light arrays 130 positioned in a distal end 815 of probe 805 to project light substantially parallel to a longitudinal axis of housing 810.

Light arrays 130, similar to those shown in FIGS. 1A and 1B previously discussed, comprise a plurality of lighting sources 135a-135h, that emit light in one or more wavelength ranges. Also shown, is electrical connector 165 that provides power and control signals to electronic components (not shown) that distribute the applied power/control signals to lighting sources 135a-135h within light bearing element 820. In this exemplary embodiment, housing 810 operates in a manner similar to sleeve 160 to confine electrical energy within electrical connector 165.

In one aspect of the invention, light bearing element 820 may be integrated onto the distal end of probe element 805. Alternatively, light bearing element 820 may be removably attachable to the distal end of probe element 805.

Further illustrated is probe sleeve 880 into which housing 810 may be inserted. Probe sleeve 880 may be removable attachable to housing 810 to allow for cleaning, or the disposal, of sleeve 880. In one aspect of the invention, sleeve 880 may be composed of a clear (transparent), a translucent or a frosted material.

FIG. 9 illustrates an expanded view of the area, designated as FIG. 9 in FIG. 8, illustrating the positioning on light arrays 130a-130d on distal end 815 of light bearing element 820. Light arrays 130a-130d, as previously discussed, comprise a plurality of lighting sources 135 (e.g., 135a-135d).

In one aspect of the invention, light arrays 130 (i.e., 130a-130d) may comprise lighting configurations similar to those shown in FIG. 1B, wherein 8 lighting sources 135 (135a-135h) are shown. However, it would be recognized by those skilled in the art that the number of light arrays 130 and the number of lighting sources 135 within each of the light arrays 130 may be increased or decreased without altering the scope of the invention.

Further illustrated is image capture device 150 positioned on distal end 815. Image capture device 150, as previously discussed, captures images of the area illuminated by lighting sources 135. In this illustrated embodiment, positioned adjacent image capture device 150 is shown emission filter 190. Emission filter 190 limits the wavelength range of the images captured by image capture device 150 to a desired wavelength range, which is based in part on the wavelength of light emitted by one or more of light sources 135a-135h and an expected wavelength response of light generated by objects (e.g., bacteria) illuminated by the light emitted by one or more of light sources 135a-135h. In a preferred embodiment of the invention, the wavelength response of emission filter 190 is configured to remove or significantly attenuate wavelengths associated with the light emitted by light sources 135a-135h, through the process of absorption or reflection, while allowing the expected wavelengths to pass unattenuated. Emission filter 190 may comprise a low pass filter, a high pass filter, a notch filter and/or a bandpass filter.

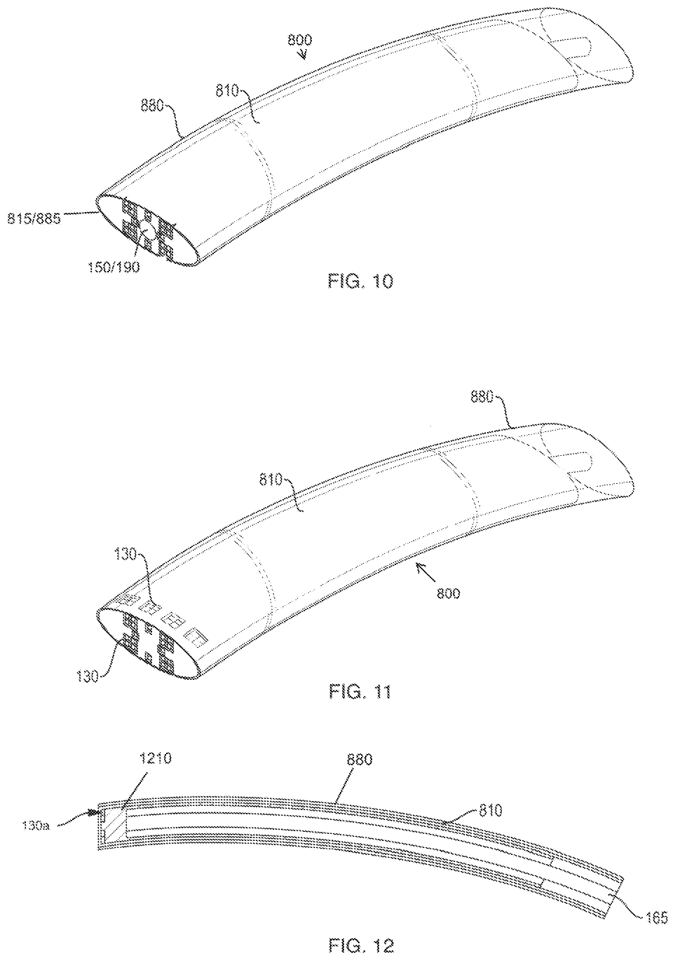

FIG. 10 illustrates a second prospective view of light therapy device 800, shown in FIG. 8, wherein housing 810 is contained within sleeve 880. Further illustrated are image capture device 150, filter 190 and light arrays 130 positioned on a distal end 815 of light bearing element 820, which emit light through distal end 885 of sleeve 880. Distal end 885 may be of an optically clear material, a translucent material or a frosted material to allow light emitted by lighting sources 135a-135h to be transmitted, therethrough.

Although, emission filter 190 is shown adjacent image capture device 150, it would be recognized that distal end 885 of sleeve 880 may comprise a material configured to include the optical characteristics of filter 190, as previously discussed. In this case, a separate emission filter 190 adjacent image capture device 150 is not needed.

FIG. 11 illustrates a prospective view of another aspect of the exemplary embodiment of light therapy device 800. In this illustrated second aspect, a plurality of light arrays 130 are positioned on an upper surface of housing 810 in addition to distal end 815. Light arrays 130 positioned on the upper surface (and/or lower surface) of housing 810 allow for the projection or emission of light to be substantially perpendicular to a longitudinal axis of light therapy device 800. Although, not shown, it would be recognized that lighting sources 135 and/or lighting arrays 130 may be positioned along the edges of housing 810 without altering the scope of the invention.

FIG. 12 illustrates a side view of the light therapy device 800 shown in FIG. 8, wherein printed circuit board 1210 is shown within housing 810. Printed circuit board 1210 may include conventional electronic components, such as resistors, capacitors, transistors, diodes, etc., and/or integrated circuits, such a Field Programmable Gate Arrays (FPGAs) and Application Specific Integrated Circuits (ASICs), forming electronic or electrical circuit or circuitry, that may be used to control an application of voltage to at least one lighting source 135a-135h within at least one light array 130a-130d. Power to the electronic components on printed circuit board 1210 may be provided through an electrical connection (as shown by electrical connector 165) to a remote power source (not shown) or by a conventional, or a rechargeable, battery (not shown) that may be contained within or attached to housing 810.

In one aspect of the invention, housing 810 may comprise a heat transferrable material (e.g., a metal, such as aluminum, cooper, etc.) 166 that may transfer heat generated by light sources 135a-135h away from a body cavity into which light therapy device 800 is positioned.

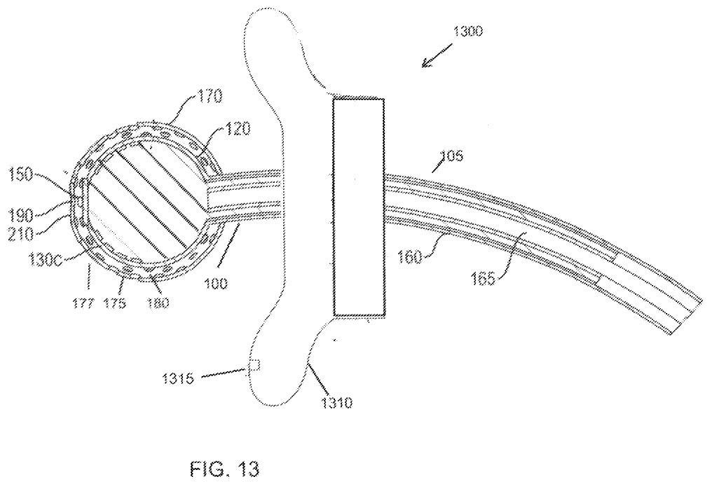

FIG. 13 illustrates a side view of still another aspect of the exemplary embodiment of the light therapy devices shown in herein.

In this illustrated embodiment light therapy device 1300, which is similar to the embodiment shown in FIG. 3, further comprises a protective mask 1310, wherein probe element 105 of light treatment device 100 is slidable with respect to mask 1310. In this illustrated example, mask 1310 is composes of a flexible, opaque, material, which is adaptable to an area around a body cavity into which light therapy device 100 is inserted. Mask 1310 prevents the escaping, and the inadvertent viewing, of light emitted by one or more of lighting sources 135a-135h. Mask 1310 may be sized to conform to an area around a user's mouth, for example, when light treatment device 100 is inserted into an oral cavity.

In accordance with one aspect of the invention, mask 1310 may include at least one sensor 1315. Sensor 1315 provides an indication that mask 1310 is in a position that prevents the inadvertent viewing of light emitted by one or more lighting sources 135a-135h. Sensor 1315 may be one of a contact sensor, a proximity sensor and a heat sensor.

Sensor 1315 operates as a safety mechanism to prevent the application of a voltage to one or more of lighting sources 135a-135h without the proper placement of mask 1310 with respect to a patient to which light therapy is applied.

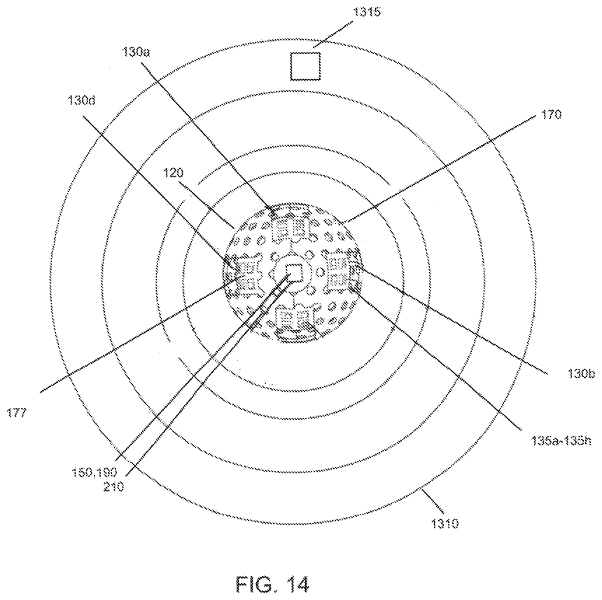

FIG. 14 illustrates a front view of the aspect of the exemplary embodiment of the light therapy device shown in FIG. 13.

In this illustrated embodiment, light therapy device 1300 includes a light bearing element 120 comprising lighting arrays 130a-130d, which comprise lighting sources 135a-135h. Further illustrated is sensor 1315 incorporated into mask 1310, wherein sensor 1315 provides an indication of proper placement of device 1300 with respect to a patient. In addition, light bearing element 120 may be extended into a body cavity by the slidable arrangement of probe 105 with respect to mask 1310.

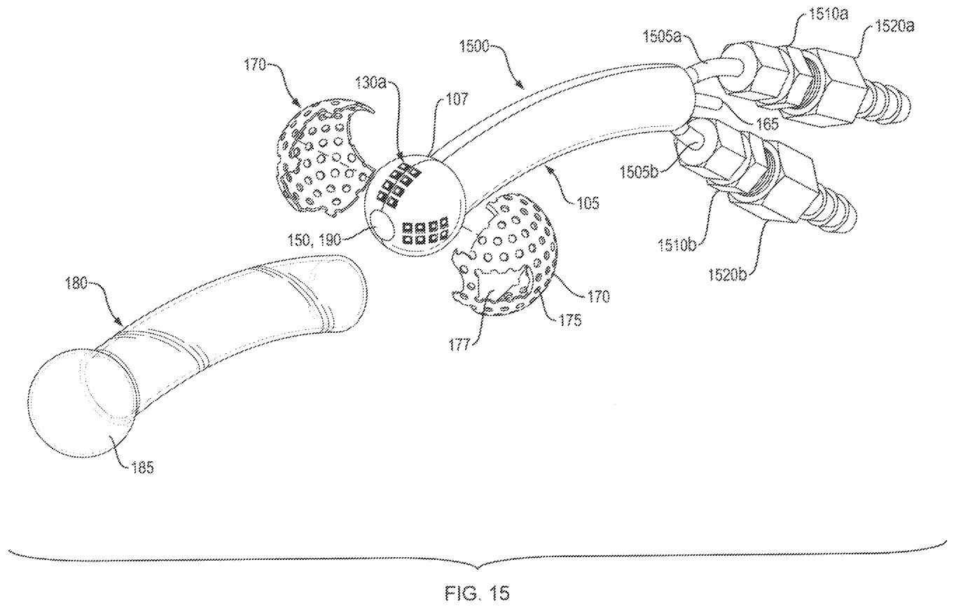

FIG. 15 illustrates an exploded perspective view of a second aspect of the first exemplary embodiment of the light therapy device shown in FIG. 1A.

Similar to the light therapy device shown in FIG. 1A, light therapy device 1500 comprises a probe body element 105 and light bearing element 120 comprising a plurality of lighting arrays 130a, 103b, etc. at a distal end 107 of body 105. Further illustrated is light distributor 170 and sleeve element 180. Sleeve element 180 is positionable over probe body element 105 and light bearing element 120, as previously discussed.

In accordance this second aspect of the invention light therapy device 1500 incorporates, therein, a cooling mechanism suitable for drawing heat generated by lighting sources 135a, 135b, etc. The drawing of heat from the lighting sources 135a, 135b, etc. allows for the insertion of light therapy device 1500 within a body cavity for a desired period of time as the temperature of probe 105 is maintained at a substantially constant and suitable temperature.

As previously discussed, a heat conductive path 166 comprising a heat transferable material may be utilized to draw heat from lighting sources 135a, 135b, etc. In addition, heat conductive path 166 may further comprise a heat transferable material (i.e., a passive heat transferable element, not shown) positioned substantially adjacent lighting sources 135a, 135b, etc., at distal end 107.

In accordance with the aspect of the invention illustrated, fluid carrying elements (e.g., pipes, conduits, etc.) 1505a, 1505b are contained within probe 105, such that a cooling fluid (e.g., air, water, etc.) may be provided to distal end 107 of probe 105 to capture and transmit heat away from the lighting sources 135a, 135b, etc., or the heat transferrable material 166 within probe 105.

In this illustrated embodiment, conduits 1505a, 1505b are connected to fittings 1510a, 1510b, wherein fittings 1510a/1520a and 1510b/1520b provide for the removable connection of light therapy device 1500 from a source (not show) of cooling fluid.

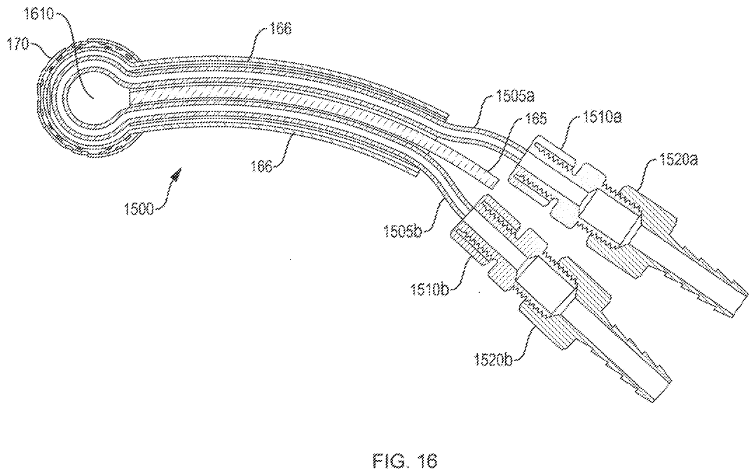

FIG. 16 illustrates a side view of the light therapy device shown in FIG. 15.

In this illustrated aspect of the invention, light bearing element 120 incorporates a heat-transferable material 1610, therein. As discussed, heat-transferable material 1610, which is positioned substantially adjacent (i.e., in contact with or in close proximity to) lighting sources 135a, 135b, etc., is configured to capture and retain heat generated by lighting sources 135a, 135b, etc. Heat transferable material 1610 provide a relatively large mass suitable for the collection of heat generated by the lighting elements 135a, 135b, etc., positioned on (or within) light bearing element 120. Further illustrated are conduits 1505a, 1505b, extending, as a single entity (i.e., in the form of a loop), around material 1610. In accordance with the principles of the invention, cooling fluid, provided by a cooling source (not shown), entering conduit 1505a, for example, may be directed around material 1610, where heat, generated by lighting sources 135a, 135b, and collected within material 1610, is transferred to the circulating cooling fluid. The heated cooling fluid may then be returned, via conduit 1505b to a cooling source (not shown).

In another aspect of the invention, heat transferrable material 166 may be positioned about conduits 1505a, 1505b to allow for additional surface area for the removal of heat generated by light sources 135a, 135b, etc.

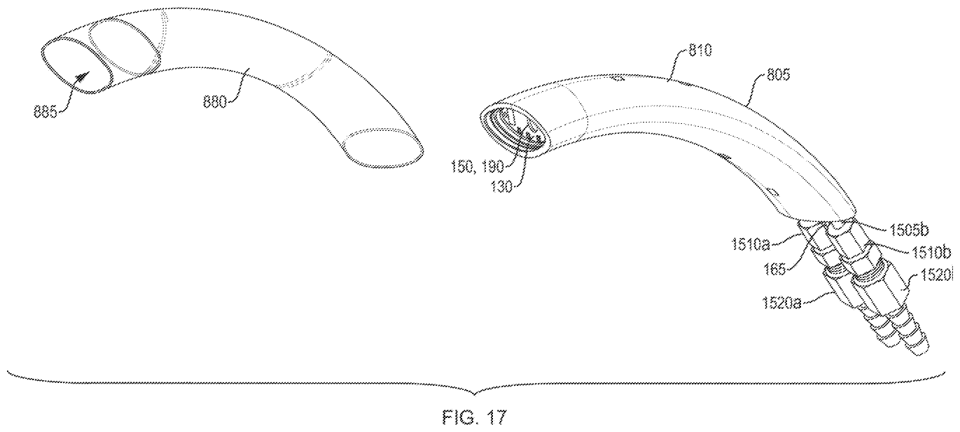

FIG. 17 illustrates an exploded perspective view of a second aspect of the second exemplary embodiment of a light therapy device in accordance with the principles of the invention.

Light therapy device 1700, similar to light therapy device 800 shown in FIG. 8, comprises a probe element 805, in the form of a housing 810, which is shown as possessing an elongated cross-sectional shape, and a light bearing element 820 attached to a distal end of housing 810. Further illustrated is sleeve 880, with distal end 885, which is slidable with respect to housing 810. Further shown is lighting array(s) 130 and camera/filter combination 150/190 positioned on distal 815, as previously discussed.

In accordance with this aspect of the invention, conduit 1505b (only a single conduit shown), provides cooling fluid to (or returns cooling fluid from) lighting sources 135a, 135b on the distal end 815 of probe 810, in a manner similar to that discussed with regard to FIGS. 15 and 16. Further illustrated is fittings 1510a, 1510b, (which are attached to conduits 1505a, 1505b, respectively) that are removably attached to fittings 1520a, 1520b. The combination of fittings 1510a/1520a and 1510b/1510b provide for the removable attachment of probe 1700 with respect to a cooling source (not shown), as previously discussed.

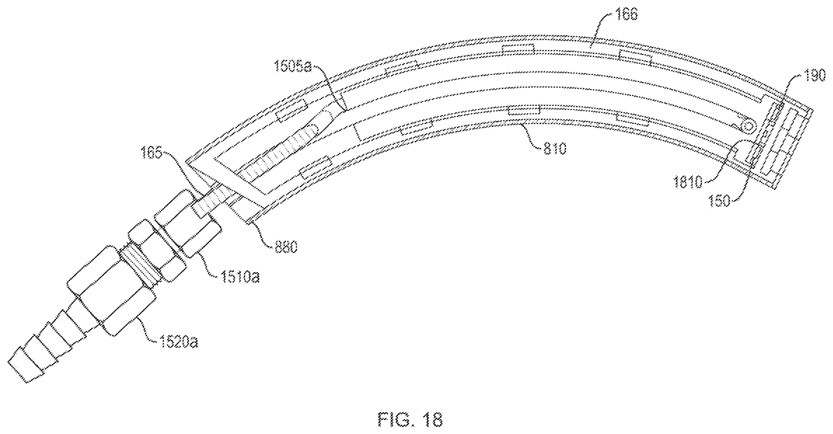

FIG. 18 illustrates a side view of the light therapy device shown in FIG. 17.

In accordance with the principles of the invention, conduits 1505a, 1505b (of which 1505b is not shown) extend, as a continuous loop, from a proximal end of device 1700 to a distal end of device 1700 such that cooling fluid may be provided to a heat collection region 1810 proximate (e.g., in contact with or substantially adjacent) to the lighting elements 135a, 135b, etc., to transfer heat, generated by lighting elements 135a, 135b, etc., away from the lighting elements 135a, 135b, etc.

In accordance with one aspect of the invention, conduits 1505a, 1505b may pass through or along passive heat collecting element 166, which further allows for the collection and distribution of heat generated by the lighting sources 135a, 135b, etc. Similar to heat transfer element 1610 (FIG. 16), heat transfer element 1810 may comprise an element such as copper or aluminum or similar materials or combinations, thereof, to expedite the transfer of heat from lighting sources 135a, 135b, etc. In addition, or alternatively, light therapy device 1700 may include a heat transferrable material 166 in close proximity or adjacent conduits 1505a, 1505b to further expedite the transfer of heat away from lighting sources 135a, 135b, etc.

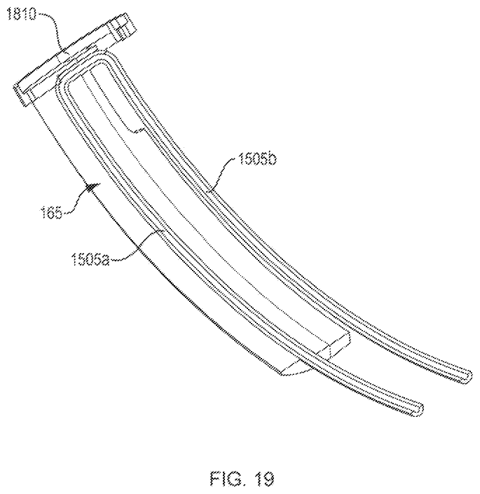

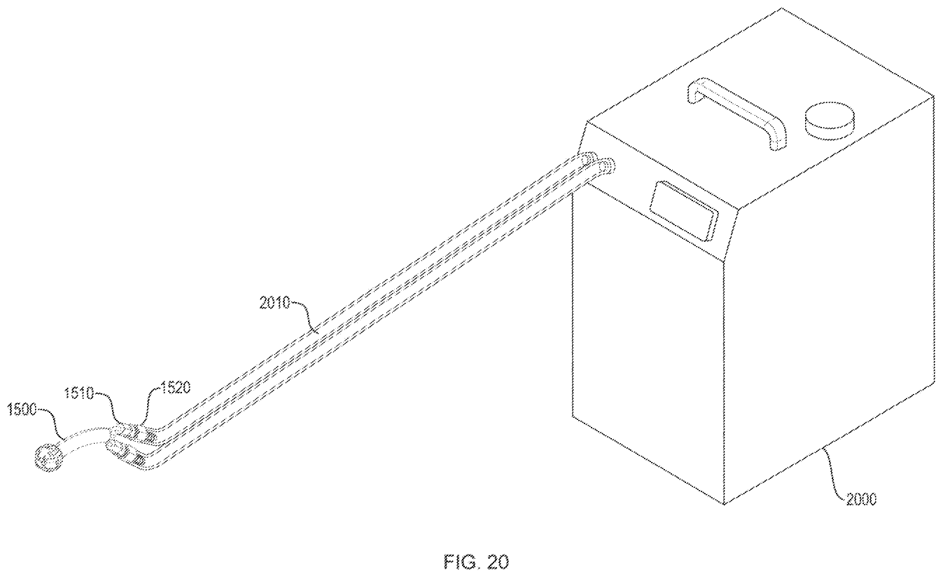

FIG. 19 illustrates a perspective view of an embodiment of a loop formation of conduits 1505a,1505b, which provides cooling fluid to an area substantially adjacent to lighting elements 135a, 135b, etc., and draws, heat generated by the lighting elements 135a, 135b, etc., away. Further illustrated is passive heat collecting elements 166 and 1810, which allow for the withdrawal of heat from the lighting elements 135a, 135b, etc.

FIG. 20 illustrates an exemplary cooling element 2000 suitable for providing a cooling fluid (air, water, etc.) through conduits 2010 to probe element 1500 (or 1700), wherein the cooling fluid is transferred through fittings 1510a/1520a, 1510b/1520b.

In one aspect of the invention, cooling element 2000 may include a temperature sensor, which monitors the returned heating cooling fluid temperature to determine the level of cooling achieved. In one aspect of the invention, the temperature of the cooling fluid provided to light therapy device 1500 (1700) may be determined, in part, based on the temperature of the returned cooling fluid temperature. For example, the temperature of the provided cooling fluid may be decreased when the temperature of the returned fluid is greater than a desired value. Alternatively, the temperature of the cooling fluid may be increased when the temperature is below a desired value. Accordingly, the temperature of the light therapy device is maintained at a temperature that is compatible (i.e., not too hot, not too cold) with the body cavity. The maintenance of the temperature allows for a long-term insertion of the light therapy devices shown, herein, in the body cavity.

Although FIGS. 13 and 14 illustrates a configuration wherein mask 1310 is applied to the embodiment of the invention shown in FIG. 3, it would be recognized that mask 1310 may be applicable to the embodiments of the invention shown in FIGS. 6, 12, 15 and 17, without altering the scope of the invention claimed.

In summary, a light therapy device is disclosed that is suitable for insertion into body cavity, which provides for the identification of bacterial and/or viral infection within the body cavity, and providing for a photo-basis treatment of the identified bacterial and/or viral infection, wherein the light therapy device includes a plurality of lighting sources arranged in light arrays that emit light when inserted into the body cavity, the plurality of lighting sources emitting light for the identification and the treatment of bacterial and/or viral infection. And further disclosed is a heat transfer system that provides for the withdrawal of heat generated by lighting sources, wherein light therapy device may be maintained at a nominal temperature so as to retain the light therapy device within a body cavity for a desired period of time

Although the invention disclosed herein discusses specific wavelengths that are produced with currently available LEDs (i.e., non-lasing light emitting diodes and laser diodes), it would be recognized that the specific wavelengths absorbed and/or reflected may be changed and/or added to without altering the scope of the invention. In addition, it would be known in the art that the specific wavelengths discussed, herein, represent a band of wavelengths centered on the wavelength values presented herein to account for divergence of the wavelength generated by the LED during the generation of the light and/or the operation of the LED, wherein the light generated is represented as a nominal value.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

1. A light therapy device insertable within a body cavity, said light therapy device comprising:
a probe extending from a proximal end to a distal end, said probe comprising:
an electrical connector extending from said proximal end to said distal end; and
a heat transfer element, extending from said proximal end to said distal end;
a light bearing element, attached to said distal end, said light bearing element comprising:
a plurality of lighting elements arranged on a surface of said light bearing element, wherein said electrical connector is configured to:
provide power and control signals to said plurality of lighting elements; and
said heat transfer element is configured to:
draw heat generated by said plurality of lighting elements to said proximal end of said probe;
a light distributor surrounding at least said light bearing element, said light distributor, removably attachable to said light bearing element, being configured to:
allow light, emitted by selected ones of said plurality of lighting elements, to illuminate substantially adjacent to selected tissue within said body cavity.

2. The light therapy device of claim 1, wherein said light bearing element is one of:
integrated onto said distal end of said probe and removably attachable to said distal end of said probe.

3. The light therapy device of claim 1, wherein said light bearing element is one of:
a spherical shape, an elliptical shape and an elongated shape.

4. The light therapy device of claim 1, wherein said light distributor comprises at least one of:
an optical clear material and an opaque material, wherein said opaque material comprises at least one of: a plurality of openings and a plurality of perforations.

5. The light therapy device of claim 1, comprising:
an image capture device positioned on a distal end of said light bearing device.

6. The light therapy device of claim 1, comprising:
a probe sleeve removably attachable to said light therapy device.

7. The light therapy device of claim 6, wherein said probe sleeve is positioned within said light distributor.

8. The light therapy device of claim 6, wherein said light distributor is positioned within said probe sleeve.

9. The light therapy device of claim 1, wherein said plurality of lighting elements emit light in at least one of: an ultra-violet wavelength range, a visible wavelength range and an infra-red wavelength range.

10. The light therapy device of claim 5, comprising:
a filter, positioned adjacent said image capture device, said filter configured to:
attenuate light in at least one of wavelength range emitted by said plurality of lighting elements and pass light in a second light wavelength range, wherein said second light wavelength range is based at least on said light emitted by said plurality of lighting elements.

11. The light therapy device of claim 6, wherein said probe sleeve comprises:
a filter aligned with said image capture device.

12. The light therapy device of claim 6, wherein said probe sleeve is composed of one of:
an optically clear material, a translucent material, and a frosted material.

13. The light therapy device of claim 1, wherein said light distributor comprises:
an opaque material; and
at least one of: a plurality of openings and a plurality of perforations, within said opaque material.

14. The light therapy device of claim 1, wherein said heat transfer element comprises:
a distal end heat transfer component positioned substantially adjacent said lighting elements.

15. The light therapy device of claim 1, wherein said probe comprises one of:
a rigid material, a pliable material and a flexible material.

16. The light therapy device of claim 1, comprising:
a mask, wherein said probe is slidably with respect to said mask.

17. The light therapy device of claim 16, wherein said mask comprises:
a switch, said switch configured to:
provide an indication said mask is appropriately positioned with respect to said body cavity.

18. The light therapy device of claim 17, wherein said switch is one of:
a contact switch, a proximity switch and a heat sensor.

19. The light therapy device of claim 14, wherein said heat transfer element comprises:
a conduit arranged in a loop, said conduit configured to:
conduct a cooling fluid.

20. The light therapy device of claim 19, wherein said loop is substantially adjacent said distal end heat transfer component.
